(19) United States
(12) Patent Application Publication
(10) Pub. No.: US 2012/0029027 A1
(43) Pub. Date: Feb. 2, 2012

(54) N-[(6-AZA-BICYCLO[3.2.1]OCT-5-YL)-ARYL-METHYL]-HETEROBENZAMIDE DERIVATIVES, PREPARATION THEREOF, AND THERAPEUTIC USE OF SAME

(75) Inventors: Geneviève Estenne-Bouhtou, Paris (FR); Florence Medaisko, Paris (FR)

(73) Assignee: SANOFI, Paris (FR)

(21) Appl. No.: 13/256,834

(22) PCT Filed: Mar. 15, 2010
(86) PCT No.: PCT/FR2010/050448

§ 371 Date: Oct. 3, 2011

(30) Foreign Application Priority Data
Mar. 16, 2009 (FR) 09/01220

(51) Int. Cl. A61K 31/403; C07D 401/12; A61K 31/4439; A61P 25/28; A61K 31/422; A61P 25/00; A61P 25/18; A61P 25/24; C07D 409/12; C07D 413/12

(52) U.S. Cl. 514/339; 548/468; 514/414; 546/276.7; 548/247; 514/378

(57) ABSTRACT

The invention relates to a compound of the general formula (I), where: R is a hydrogen atom or a group selected from the (C₁-C₆)alkyl or (C₃-C₇)-cycloalkyl groups, optionally substituted by one or more groups independently selected from a halogen atom anti the (C₃-C₇)-cycloalkyl, (C₁-C₆)alkyl, (C₁-C₆)alkoxy, and hydroxy groups; R₁ is a phenyl group optionally substituted by one or more substituents independently selected from halogen atoms and the (C₁-C₆)alkyl, (C₁-C₆)alkoxy, halo-(C₁C₆)alkyl, hydroxy, halo-(C₁-C₆)alkoxy, (C₁-C₆)alkyl-thio, (C₁-C₆)alkyl-SO, and (C₁-C₆)alkyl-SO₂ groups; R₂ is one or more substituents selected from a hydrogen atom, halogen atoms, and the halo-(C₁-C₆)alkyl, (C₁-C₆)alkyl, (C₃-C₇)cycloalkyl, (C₃-C₇)-cycloalkyl-(C₁C₃)alkyl, phenyl, benzyl, (C₁-C₆)alkoxy, (C₁-C₆)alkyl-thio, (C₁-C₆))alkyl-SO, and (C₁-C₆)alkyl-SO₂ groups; and Het is a heteroaryl group; wherein said compound is in the form of a base or an acid addition salt. The invention also relates to the therapeutic use thereof and to a method for synthesising same.

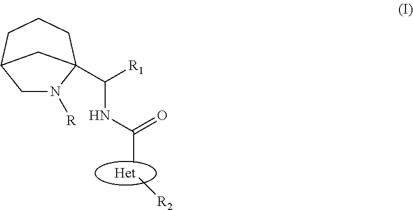

The present invention relates to derivatives of N-[(6-azabicyclo[3.2.1]oct-5-yl)arylmethyl]heterobenzamide, to the preparation thereof and to the therapeutic use thereof, in the treatment or prevention of diseases involving GlyT1 glycine transporters.

The compounds of the invention correspond to the general formula (I):

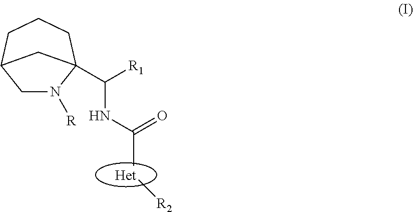

in which:
R represents a hydrogen atom or a group chosen from (C₁-C₆)alkyl or (C₃-C₇)-cycloalkyl groups, which is optionally substituted with one or more groups chosen independently from one another from a halogen atom, and (C₃-C₇)cycloalkyl, (C₁-C₆)alkyl, (C₁-C₆)alkoxy or hydroxy groups;
R₁ represents a phenyl group, optionally substituted with one or more substituents chosen, independently from one another, from halogen atoms, and (C₁-C₆)alkyl, (C₁-C₆)alkoxy, halo(C₁-C₆)alkyl, hydroxy, halo(C₁-C₆)alkoxy, (C₁-C₆)alkylthio, (C₁-C₆)alkyl-SO or (C₁-C₆)alkyl-SO₂ groups;
Het represents a heteroaryl group;
R₂ represents one or more substituents chosen from a hydrogen atom, halogen atoms and halo(C₁-C₆)alkyl, (C₁-C₆)alkyl, (C₃-C₇)cycloalkyl, (C₃-C₇)cycloalkyl(C₁-C₃)alkyl, phenyl, benzyl, (C₁-C₆)alkoxy, (C₁-C₆)alkylthio, (C₁-C₆)alkyl-SO and (C₁-C₆)alkyl-SO₂ groups;
   in the form of a base or addition salt with an acid.

The compounds of formula (I) comprise three asymmetric carbon atoms. They may therefore exist in the form of diastereoisomers and enantiomers. These enantiomers, including racemic mixtures, are part of the invention.

The compounds of formula (I) may exist in the form of bases or addition salts with acids. Such addition salts are part of the invention.

These salts are advantageously prepared with pharmaceutically acceptable acids, but salts of other acids that are useful, for example, for the purification or isolation of the compounds of formula (I) are also part of the invention.

Within the context of the invention, the following definitions apply:

Cₜ-C_{z} where t and z may take the values of 1 to 6: a carbon-based chain which may have from t to z carbon atoms, for example C₁-C₆ is a carbon-based chain which may have from 1 to 6 carbon atoms;
alkyl: a linear or branched saturated aliphatic group; for example a C₁-C₆-alkyl group represents a linear or branched carbon-based chain having from 1 to 6 carbon atoms, for example a methyl, ethyl, propyl, isopropyl, butyl, isobutyl, tert-butyl, pentyl or hexyl;
alkenyl: a linear or branched monounsaturated or polyunsaturated aliphatic group comprising, for example, one or two ethylenically unsaturated groups;
amino: an NH₂ group;
alkoxy: an —O-alkyl group;
hydroxy: an —OH group;
alkylthio: a sulphur atom substituted with an alkyl group;
halogen atom: a fluorine, a chlorine, a bromine or an iodine;
haloalkyl; an alkyl group of which one or more hydrogen atoms have been substituted with a halogen. By way of example, mention may be made of trifluoromethyl, trifluoroethyl or pentafluoroethyl groups; and
heteroaryl group: a 5- or 10-membered heteroaromatic monocyclic or bicyclic group comprising from 1 to 3 heteroatoms chosen from nitrogen, oxygen and sulphur. By way of example of a heteroaryl group, mention may be made of pyrrole, furan, thiophene, pyrazole, imidazole, triazole, tetrazole, oxazole, isoxazole, oxadiazole, thiazole, isothiazole, thiadiazole, pyridine, pyrimidine, pyrazine, pyridazine, triazine, indole, isoindole, benzimidazole, indazole, indolizine, benzofuran, isobenzofuran, benzothiophene, benzo[c]thiophen, pyrrolopyridine, imidazopyridine, pyrazolopyridine, triazolopyridine, tetrazolopyridine, pyrrolopyrimidine, imidazopyrimidine, pyrazolopyrimidine, triazolopyrimidine, tetrazolopyrimidine, pyrrolopyrazine, imidazopyrazine, pyrazolopyrazine, triazolopyrazine, tetrazolopyrazine, pyrrolopyridazine, imidazopyridazine, pyrazolopyridazine, triazolopyridazine, tetrazolopyridazine, pyrrolotriazine, imidazotriazine, pyrazolotriazine, triazolotriazine, tetrazolotriazine, furopyridine, furopyrimidine, furopyrazine, furopyridazine, furotriazine, oxazolopyridine, oxazolopyrimidine, oxazolopyrazine, oxazolopyridazine, oxazolotriazine, isoxazolopyridine, isoxazolopyrim idine, isoxazolopyrazine, isoxazolopyridazine, isoxazolotriazine, oxadiazolopyridine, oxadiazolopyrimidine, oxadiazolopyrazine, oxadiazoiopyridazine, oxadiazolotriazine, benzoxazole, benzisoxazole, benzoxadiazole, thienopyridine, thienopyrimidine, thienopyrazine, thienopyridazine, thienotriazine, thiazolopyridine, thiazolopyrim idine, thiazolopyrazine, thiazolopyridazine, thiazoiotriazine, isothiazolopyridine, isothiazolopyrimidine, isothiazolopyrazine, isothiazolopyridazine, isothiazolotriazine, thiadiazolopyridine, thiadiazolopyrimidine, thiadiazolopyrazine, thiadiazolopyridazine, thiadiazolotriazine, benzothiazole, benzoisothiazole, benzothiadiazole, quinoline, isoquinoline, cinnoline, phthalazine, quinoxaline, quinazoline, naphthyridine, benzotriazine, pyridopyrimidine, pyridopyrazine, pyridopyridazine, pyridotriazine, pyrimidopyrimidine, pyrimidopyrazine, pyrimidopyridazine, pyrimidotriazine, pyrazinopyrazine, pyrazinopyridazine, pyrazinotriazine, pyridazinopyridazine and pyridazinotriazine groups.

Among the compounds of general formula (I) that are subjects of the invention, a first group of compounds is constituted by the compounds for which:
R₁ represents a phenyl group optionally substituted with one or more substituents chosen, independently from one another, from halogen atoms, (C₁-C₆)alkyl or halo(C₁-C₆)alkyl groups;
R, Het and R₂ being as defined above.

Among the compounds of general formula (I) that are subjects of the invention, a second group of compounds is constituted by the compounds for which:
Het represents an imidazole, isoxazole, indole, thiophene or pyridine group;
R, R₁, and R₂ being as defined above.

Among the compounds of general formula (I) that are subjects of the invention, a third group of compounds is constituted by the compounds for which:
R₂ represents one or more substituents chosen from a hydrogen atom, halogen atoms, halo(C₁-C₆)alkyl, (C₁-C₆)alkyl, phenyl, benzyl, (C₁-C₆)alkoxy or (C₁-C₆)alkylthio groups;
R₁, Het and R₁ being as defined above.

Among the compounds of general formula (I) that are subjects of the invention, a fourth group of compounds is constituted by the compounds for which:
R₁ represents a phenyl group optionally substituted with one or more substituents chosen, independently from one another, from halogen atoms, (C₁-C₆)alkyl or halo(C₁-C₆)alkyl groups;
Het represents an imidazole, isoxazole, indole, thiophene or pyridine group;
R₂ represents one or more substituents chosen from a hydrogen atom, halogen atoms, halo(C₁-C₆)alkyl, (C₁-C₆)alkyl, phenyl, benzyl, (C₁-C₆)alkoxy or (C₁-C₆)alkylthio groups.

Among the compounds of general formula (I) that are subjects of the invention, a fifth group of compounds is constituted by the compounds for which:
R₁ represents a phenyl group optionally substituted with one or more substituents chosen, independently from one another, from fluorine atoms, methyl or trifluoromethyl groups;
Het represents an imidazole, isoxazole, indole, thiophene or pyridine group;
R₂ represents one or more substituents chosen from a hydrogen atom, chlorine atoms, methyl, methoxy, trifluoromethyl, methylthio, phenyl or benzyl groups.

The combinations of groups one to five as defined above are also part of the invention.

Among the compounds of general formula (I) that are subjects of the invention, mention may especially be made of the following compounds:
N-[(6-Azabicyclo[3.2.1]oct-5-yl)phenylmethyl](2,5-dichloro)thiophene-3-carboxamide;
N-[(6-Azabicyclo[3.2.1]oct-5-yl)phenylmethyl]-2-methylsulfanynicotinamide;
N-[(6-Azabicyclo[3.2.1]oct-5-yl)phenylmethyl](3-chloro-4-trifluoromethyl)pyridine-2-carboxamide and its hydrochloride;
N-[(6-Azabicyclo[3.2.1]oct-5-yl)phenylmethyl](5-methyl-3-phenyl)isoxazole-4-carboxamide;
N-[(6-Azabicyclo[3.2.1]oct-5-yl)phenylmethyl](1-benzyl-2-ethyl-5-methoxy)-1H-indole-3-carboxamide;
[(6-Azabicyclo[3.2.1]oct-5-yl)phenylmethyl](1-benzyl)-1H-indole-4-carboxamide;
N-[(6-Azabicyclo[3.2.1]oct-5-yl)phenylmethyl](1-methyl)-1H-imidazole-4-carboxamide;
N-[(6-Azabicyclo[3.2.1]oct-5-yl)(4-fluorophenyl)methyl]-2-methylsulfanyl-nicotinamide and its hydrochloride;
N-[(6-Azabicyclo[3.2.1]oct-5-yl)(4-fluorophenyl)methyl](3-chloro-4-trifluoromethyl)-pyridine-2-carboxamide and its hydrochloride;
N-[(-6-Azabicyclo[3.2.1]oct-5-yl)-m-tolylmethyl]-2-methylsulfanylnicotinamide and its hydrochloride;
N-[(6-Azabicyclo[3.2.1]oct-5-yl)(3-trifluoromethylphenyl)methyl](3-chloro-4-trifluoromethyl)pyridine-2-carboxamide and its hydrochloride.

The compounds of the invention have a particular activity as inhibitors of GlyT1 glycine transporters, especially improved activity and safety profiles.

The compounds of general formula (I) may be prepared by a process illustrated by scheme 1 below:

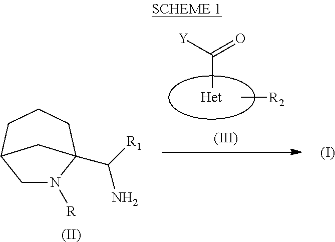

A diamine of general formula (II), in which R and R₁ are as defined above, especially when R represents a hydrogen atom, is coupled with an activated acid, for example via a mixed anhydride or an acid chloride of general formula (III) in which Y represents a leaving group derived, for example, from benzotriazole, acylurea or a halogen atom and R₂ is as defined above, using the methods known to a person skilled in the art.

The compounds of general formula (I) in which R represents a hydrogen atom may also be prepared from compounds of general formula (I) in which R represents a protective group which can be deprotected via hydrogenolysis.

The compounds of general formula (I) in which R is other than a hydrogen atom may also be prepared starting from compounds of general formula (I) in which R represents a hydrogen atom, either by alkylation with a halide or mesylate of RX type, in which R is as defined above and X is a mesylate or a halogen atom, in the presence of a mineral base, for example potassium carbonate in acetonitrile, or via an Eschweiler-Clarke reaction or via a reductive amination with a suitable aldehyde or ketone according to the methods known to a person skilled in the art, or with a suitable epoxide derivative according to the methods known to a person skilled in the art, or with a suitable epoxide derivative according to the methods known to a person skilled in the art.

The compounds of general formula (I) in which the R₁ group is a phenyl group substituted with a hydroxy may be obtained from the corresponding compound of general formula (I) substituted with a methoxy, by using the methods known to a person skilled in the art,

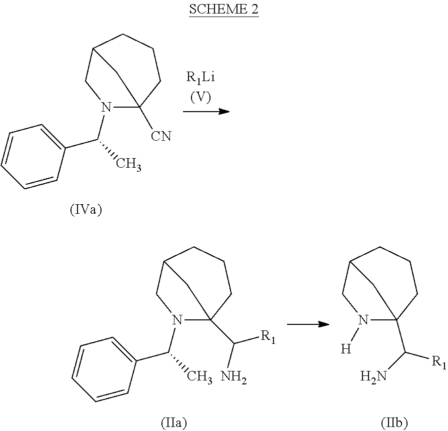

According to scheme 2, the nitrile of formula (IVa) is reacted with the lithiated aromatic compound of general formula (V), in which R₁ is as defined above, in an ethereal solvent such as tetrahydrofuran or ether, at low temperature, for example at −70° C. An imine is thus obtained, which is, in particular, diastereoselectively reduced with a reducing agent such as sodium borohydride in a protic solvent such as methanol in order to give the amine of general formula (IIa). The amine (IIa) may be debenzylated by hydrogenation in the presence of a palladium catalyst in order to provide the deprotected amine (IIb) (Scheme 2).

Furthermore, the chiral compounds of general formula (I) may be obtained by separation of the racemic compounds via high performance liquid chromatography (HPLC) on a chiral column, or by separation, via silica gel chromatography, of the chiral diastereoisomers of the amine of general formula (IIa) then debenzylation, as described in scheme 2.

The nitrite of formula (IVa) is prepared according to a method described in Tetrahedron: Asymmetry, 2006 (17), 252-258.

The lithiated aryl compounds of general formula (V) may be prepared according to methods known to a person skilled in the art.

The acids and acid chlorides of general formula (III) are commercially available or are prepared by analogy with methods known to a person skilled in the art.

The following examples illustrate the preparation of some compounds of the invention. In these examples:
The elemental microanalyses, the IR and NMR spectra and the chiral column HPLC confirm the structures and the enantiomeric purities of the compounds obtained.
For the NMR descriptions, “m” stands for multiplet, “s” singlet, “t” triplet, “d” doublet, “q” quadruplet, dxd stands for double doublet, txt stands for triple triplet, dxt double triplet, etc.
The numbers indicated between parentheses in the titles of the examples correspond to those from the first column of the table given below.
The term “decomp.” stands for “decomposition”.
“ee” stands for enantiomeric excess;
The roman numerals between parentheses correspond to the corresponding general formulae which are indicated in the synthesis schemes.
The nomenclature used is the nomenclature according to the IUPAC (International Union of Pure and Applied Chemistry) recommendations.

In the names of the compounds, the dash “-” is part of the word and the dash “_” is only used for the break at the end of the line; it should be deleted in the absence of a break and should not be replaced by a normal dash nor by a space.

### EXAMPLE 1

### (Compound No. 2): N-[(6-azabicyclo[3.2.1]oct-5-yl)phenylmethyl](2-methylsulphanyl)nicotinamide

1.1 Phenyl-[6-((R)-1-phenylethyl)-6-azabicyclo[3.2.1]oct-5-yl]methylamine.

Placed in a 100 ml three-necked flask, under argon, is 1 g of 6-((R)-1-phenylethyl)-6-azabicyclo[3.2.1]octane-5-carbonitrile (IVa) (4.16 mmol) at −70° C. in 35 ml of anhydrous tetrahydrofuran. 7.4 ml of a 1.13M solution (cyclohexane/ether) of phenyllithium (8.32 mmol) are added dropwise.

The mixture is left for two and a half hours at −70° C., then hydrolysed at −20° C. with 15 ml of water.

After extraction, the organic phase is concentrated under reduced pressure, then the residue is taken up in 20 ml of methanol. 0.79 g of sodium borohydride (20.8 mmol) is added thereto in portions. The reaction medium is left stirring overnight at ambient temperature.

After evaporation under reduced pressure, the residue is taken up with 50 ml of ether and 50 ml of water. The medium is acidified with a 1N hydrochloric acid solution, then extracted. The aqueous phase is basified with aqueous ammonia then re-extracted two times with 50 ml of dichloromethane. The organic phases are combined, dried over sodium sulphate, filtered and evaporated under reduced pressure. Thus 1.5 g of an oil is obtained, which is purified by chromatography over a silica gel column by eluting with a mixture of dichloromethane and methanol. Thus, 115 g of phenyl-[6-((R)-1-phenylethyl)-6-azabicyclo[3.2.1]oct-5-yl]methylamine are obtained, a mixture of 2 chiral diastereoisomers, in oil form.

¹H NMR (400 MHz, DMSO-d6) δ ppm 7.6-7.10 (m, 10H), 4.20 (2.5H), 3.60 (0.5H).

### 1.2 (6-Azabicyclo[3.2.1]oct-5-yl)phenylmethylamine.

Placed in a Parr flask are 4 g of a compound of formula (IIa) (12.5 mmol) in 80 ml of methanol in the presence of a spatula tipful of 20% palladium hydroxide under 4 atmospheres of hydrogen at ambient temperature for 6 hours.

After filtration of the catalyst and evaporation of the filtrate under reduced pressure, the residue is taken up with 10 ml of dichloromethane and 20 ml of aqueous ammonia. After extraction, the organic phase is washed in a saturated solution of sodium chloride, dried over sodium sulphate, filtered, then the solvent is evaporated under reduced pressure. Thus, 1 g of (6-azabicyclo[3.2.1]oct-5′ yl)phenylmethylamine is obtained in oil form, which may be used crude in the subsequent step.

An analytical sample is obtained by salification of the base with a 2N hydrochloric ether solution then trituration in ether.

MP=215-225° C.

¹H NMR (400 MHz, DMSO-d6) δ ppm 9.14 (m, 4H), 7.76 (m, 2H), 7.56-7.43 (m, 4H), 5.09 (broad s, 1H), 3.49 (m, 1H), 3.11 (m, 1H), 2.72 (m, 1H), 2.19 (m, 1H), 1.87 (m, 1H), 1.83-1.37 (m, 8H).

### 1.3 N-[(6-azabicyclo[3.2.1]oct-5-yl)phenylmethyl](2-methylsulphanyl)nicotinamide.

Placed in a 25 ml round-bottomed flask are 155 mg of (2-methylsulphanyl)nicotinic acid (0.92 mmol), 124 mg of hydroxybenzotriazole (0.92 mmol) and 180 mg of 1-[3-(dimethylamino)propyl]-3-ethylcarbodiimide hydrochloride (0.92 mmol) in solution in 5 ml of dichloromethane and the mixture is stirred at ambient temperature for 15 minutes. 200 mg (0.92 mmol) of (6-azabicyclo[3.2.1]oct-5-yl)phenylmethylamine in solution in 5 ml of dichloromethane are added and the mixture is stirred at ambient temperature overnight.

The reaction medium is then diluted with 10 ml of dichloromethane, then washed successively with water (5 ml) with 1N sodium hydroxide (5 ml) and with a saturated solution of sodium chloride (5 ml).

The organic phase is dried over sodium sulphate, filtered and evaporated under reduced pressure.

The residue is purified by chromatography over a silica gel column by eluting with a mixture of dichloromethane and methanol. Thus, 108 mg of N-[(6-azabicyclo[3.2.1]oct-5-yl)phenylmethyl](2-methylsulphanyl)nicotinamide are obtained, in the form of a powder.

¹H NMR (400 MHz, CDCl₃) δ ppm 8.51 (dxd, J=4.8 Hz and 1.8 Hz, 1H), 7.89 (dxd, J=7.5 Hz and 1.8 Hz, 1H), 7.50-7.40 (m, 3H), 7.34-7.24 (m, 3H), 7.06 (m, 1H), 4.98 (d, J=6.8 Hz, 1H), 3.07-2.97 (m, 2H), 2.64 (s, 3H), 2.28 (m, 1H), 179-1.27 (m, 8H).

MP=138-140° C.

The other compounds listed in Table 1 are obtained according to the method described in Example 1, from the amine of formula (IIb).

Table 1 below illustrates the chemical structures of some compounds of the invention.

In the column:
“Salts”, “-” denotes a compound in base form, “HCl” denotes a hydrochloride, the number between parentheses indicates the (acid:base) ratio;
The compounds of the table are in the hydrochloride form solvated by one or more molecules of water.

In the columns R, R₁ and R₂:
“Cl” stands for chlorine;
“CH₃” stands for methyl; “C₂H₅” stands for ethyl;
“OCH₃” stands for methoxy;
“Ph” stands for phenyl;
“CF₃” stands for trifluoromethyl; and
in the column “R₂”, the number in front of the substituents indicates the position in the general formula (I).

Table 2 gives the physical properties and melting points of the compounds from Table 1.

In Table 2:
the column “m/z” indicates the molecular ion (M+H⁺) or (M⁺) observed by analysis of the products by mass spectrometry, either by LC-MS (Liquid Chromatography coupled to Mass Spectroscopy) performed on a machine of Agilent LC-MSD Trap type in positive ESI mode, or by direct introduction by MS (Mass Spectroscopy) on an Autospec M (EBE) machine using the DCI-NH₃ technique or by using the electron impact technique on a machine of Waters GCT type.

TABLE 1
(I)

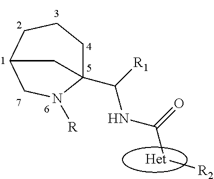

| No. | R | R₁ | 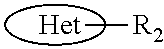 | Salts | Stereochemistry |
|---|---|---|---|---|---|
| 1 | H | Ph | 2,5-Cl₂-thiophen-3-yl | — | racemic |
| 2 | H | Ph | 2-SCH₃-pyridin-3-yl | — | racemic |
| 3 | H | Ph | 3-Cl-4-CF₃-pyridin-2-yl | HCl | racemic |
| | | | | (1:1) | |
| 4 | H | Ph | 5-CH₃-3-Ph-isoxazol-4-yl | — | racemic |
| 5 | H | Ph | 1-benzyl-2-C₂H₅-5-OCH₃- | — | racemic |
| | | | indolyl-3-yl | | |
| 6 | H | Ph | 1-benzylindolyl-4-yl | — | racemic |
| 7 | H | Ph | 1-CH₃-imidazol-4-yl | — | racemic |
| 8 | H | 4-F—Ph | 2-SCH₃-pyridin-3-yl | HCl | racemic |
| | | | | (1:1) | |
| 9 | H | 4-F—Ph | 3-Cl-4-CF₃-pyridin-2-yl | HCl | racemic |
| | | | | (1:1) | |
| 10 | H | 3-CH₃—Ph | 2-SCH₃-pyridin-3-yl | HCl | racemic |
| 11 | H | 3-CF₃—Ph | 3-Cl-4-CF₃-pyridin-2-yl | HCl | racemic |

TABLE 2

| No. | MP(° C.) | LCMS MH⁺ |
|---|---|---|
| 1 | 95-97 | 395 |
| 2 | 138-140 | 368 |
| 3 | 158-160 | 424 |
| 4 | 144-146 | 402 |
| 5 | 176-178 | 508 |
| 6 | 165-167 | 450 |
| 7 | 150-152 | 325 |
| 8 | 234-236 | 386 |
| 9 | 198-200 | 442 |
| 10 | 205-207 | 382 |
| 11 | 250.4-251.4° C. | 492 |

The compounds of the invention have been subjected to a series of pharmacological trials which have demonstrated their advantage as substances possessing therapeutic activities.

### Study of Glycine Transportation in SK-N-MC Cells Expressing the Native Human Transporter GlyT1.

The uptake of [¹⁴C]glycine is studied in SK-N-MC cells (human neuroepithelial cells) expressing the native human transporter GlyT1 by measuring the radioactivity incorporated in the presence or absence of the test compound. The cells are cultured as a monolayer for 48 hours in plates pretreated with 0.02% fibronectin. On the day of the experiment, the culture medium is removed and the cells are washed with Krebs-HEPES (4-(2-hydroxyethyl)piperazine-1-ethanesulphonic acid) buffer at pH 7.4. After preincubation for 10 minutes at 37° C. in the presence either of buffer (control batch) or of test compound at various concentrations or of 10 mM of glycine (determination of the non-specific uptake), 10 μM of [¹⁴C]glycine (specific activity 112 mCi/mmol) are subsequently added. Incubation is continued for 10 min at 37° C. and the reaction is halted by washing twice with pH 7.4 Krebs-HEPES buffer. The radioactivity incorporated by the cells is then estimated after adding 100 μl of liquid scintillant and stirring for 1 h. Counting is carried out on a Microbeta Tri-Lux™ counter. The effectiveness of the compound is determined by the IC₅₀, the concentration of the compound which reduces by 50% the specific uptake of glycine, defined by the difference in radioactivity incorporated by the control batch and the batch which received the 10 mM glycine.

The compounds of the invention have, in this test, an IC₅₀ of the order of 0.1 to 10 μM.

Table 3 indicates some examples of IC₅₀ results for compounds according to the invention.

TABLE 3

| Compound | CI₅₀ (μM) |
|---|---|
| 2 | 0.14 |
| 3 | 0.22 |
| 6 | 0.61 |
| 8 | 0.13 |
| 11 | 0.17 |

The results of the in vitro tests carried out on the compounds of the invention according to the general formula (I) show that they are inhibitors of the GlyT1 glycine transporter present in the brain.

These results suggest that the compounds of the invention may be used for treating cognitive and/or behavioural disorders associated with neurodegenerative diseases and with dementia; for treating psychoses, especially schizophrenia (deficit form and productive form) and acute or chronic neuroleptic-induced extrapyramidal symptoms; for treating various forms of anxiety, panic attacks, phobias and obsessive-compulsive disorders; for treating various forms of depression, including psychotic depression; for treating bipolar disorders, manic disorders and mood disorders; for treating disorders due to alcohol abuse or withdrawal, disorders of sexual behaviour, eating disorders and migraine disorders; pain; and sleep disorders.

The compounds according to the invention may therefore be used for preparing medicaments, in particular medicaments that are inhibitors of the GlyT1 glycine transporter.

Thus, according to another of its aspects, one subject of the invention is medicaments which comprise a compound of formula (I), or an addition salt of the latter with a pharmaceutically acceptable acid.

Another subject of the present invention is pharmaceutical compositions comprising an effective dose of at least one compound according to the invention, in the form of the base or a salt, and as a mixture, if appropriate, with suitable excipients.

Said excipients are chosen depending on the pharmaceutical form and the method of administration desired.

The pharmaceutical compositions according to the invention may thus be intended for oral, sublingual, subcutaneous, intramuscular, intravenous, topical, intratracheal, intranasal, transdermal, rectal or intraocular administration.

The unit administration forms can be, for example, tablets, gelatin capsules, granules, powders, solutions or suspensions to be taken orally or to be injected, patches or suppositories. Ointments, lotions and collyria can be envisaged for topical administration.

Said unit forms are dosed to allow a daily administration of 0.01 to 20 mg of active principle per kg of body weight, depending on the pharmaceutical dosage form.

To prepare tablets, a pharmaceutical vehicle, which can be composed of diluents, such as, for example, lactose, microcrystalline cellulose or starch, and formulation adjuvants, such as binders (polyvinylpyrrolidone, hydroxypropyl methyl cellulose, etc.), flow agents, such as silica, or lubricants, such as magnesium stearate, stearic acid, glyceryl tribehenate or sodium stearylfumarate, is added to the micronized or unmicronized active principle. Wetting agents or surfactants, such as sodium lauryl sulphate, can also be added.

The preparation techniques can be direct tableting, dry granulation, wet granulation or hot melt.

The tablets can be bare, coated with sugar, for example with sucrose, or coated with various polymers or other appropriate materials. They can be designed to make possible rapid, delayed or sustained release of the active principle by virtue of polymer matrices or of specific polymers used in the coating.

To prepare gelatin capsules, the active principle is mixed with dry pharmaceutical vehicles (simple mixing, dry or wet granulation, or hot melt) or liquid or semisolid pharmaceutical vehicles.

The gelatin capsules can be hard or soft and coated or uncoated with a thin film, so as to have a rapid, sustained or delayed activity (for example, for an enteric form).

A composition in the form of a syrup or an elixir or for administration in the form of drops can comprise the active principle in conjunction with a sweetener, preferably a calorie-free sweetener, methylparaben or propylparaben, as antiseptic, a flavour enhancer and a colorant.

The water-dispersible powders and granules can comprise the active principle as a mixture with dispersing agents or wetting agents, or dispersing agents, such as polyvinylpyrrolidone, as well as with sweeteners and flavour-correcting agents.

Recourse is had, for rectal administration, to suppositories prepared with binders which melt at the rectal temperature, for example cocoa butter or polyethylene glycols.

Use is made, for parenteral administration, of aqueous suspensions, isotonic saline solutions or injectable sterile solutions comprising pharmacologically compatible dispersing agents and/or wetting agents, for example propylene glycol or butylene glycol.

The active principle can also be formulated in the form of microcapsules, optionally with one or more supports or additives or else with a polymer matrix or with a cyclodextrin (patches or sustained release forms).

The topical compositions according to the invention comprise a medium compatible with the skin. They can be provided in particular in the form of aqueous, alcoholic or aqueous/alcoholic solutions, of gels, of water-in-oil or oil-in-water emulsions having the appearance of a cream or of a gel, of microemulsions or of aerosols or in the form of vesicular dispersions comprising ionic and/or nonionic lipids. These pharmaceutical dosage forms are prepared according to methods conventional in the fields under consideration.

By way of example, a unit administration form of a compound according to the invention in tablet form may comprise the following components:

| | | |
|---|---|---|
| Compound according to the invention | 50.0 | mg |
| Mannitol | 223.75 | mg |
| Croscaramellose sodium | 6.0 | mg |
| Maize starch | 15.0 | mg |
| Hydroxypropyl methyl cellulose | 2.25 | mg |
| Magnesium stearate | 3.0 | mg |

Via the oral route, the dose of active principle administered per day may range from 0.1 to 20 mg/kg, in one or more dosage intakes.

There may be particular cases in which higher or lower dosages are appropriate; such dosages are not outside the scope of the invention. According to standard practice, the dosage that is appropriate for each patient is determined by the physician depending on the mode of administration and the weight and response of said patient.

The present invention, according to another of its aspects, also relates to a method for treating the pathologies indicated above, which comprises the administration to a patient of an effective dose of a compound according to the invention, or a pharmaceutically acceptable salt thereof.

1. A compound of general formula (I): 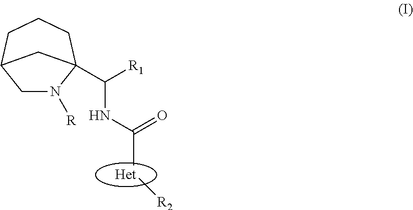
in which:
R represents a hydrogen atom or a group chosen from (C₁-C₆)alkyl or (C₃-C₇)-cycloalkyl groups, which is optionally substituted with one or more groups chosen independently from one another from a halogen atom, and (C₃-C₇)cycloalkyl, (C₁-C₆)alkyl, (C₁-C₆)alkoxy or hydroxy groups;
R₁ represents a phenyl group, optionally substituted with one or more substituents chosen, independently from one another, from halogen atoms, and (C₁-C₆)alkyl, (C₁-C₆)alkoxy, halo(C₁-C₆)alkyl, hydroxy, halo(C₁-C₆)alkoxy, (C₁-C₆)alkylthio, (C₁-C₆)alkyl-SO or (C₁-C₆)alkyl-SO₂ groups;
Het represents a heteroaryl group;
R₂ represents one or more substituents chosen from a hydrogen atom, halogen atoms and halo(C₁-C₆)alkyl, (C₁-C₆)alkyl, (C₃-C₇)cycloalkyl, (C₃-C₇)cycloalkyl(C₁-C₃)alkyl, phenyl, benzyl, (C₁-C₆)alkoxy, (C₁-C₆)alkylthio, (C₁-C₆)alkyl-SO and (C₁-C₆)alkyl-SO₂ groups;
in the form of a base or addition salt with an acid.

2. The compound of claim 1, wherein:
R₁ represents a phenyl group optionally substituted with one or more substituents chosen, independently from one another, from halogen atoms, (C₁-C₆)alkyl or halo(C₁-C₆)alkyl groups.

3. The compound of claim 1, wherein:
Het represents an imidazole, isoxazole, indole, thiophene or pyridine group.

4. The compound of claim 1, wherein:
R₂ represents one or more substituents chosen from a hydrogen atom, halogen atoms, halo(C₁-C₆)alkyl, (C₁-C₆)alkyl, phenyl, benzyl, (C₁-C₆)alkoxy or (C₁-C₆)alkylthio groups.

5. The compound of claim 1, wherein:
R₁ represents a phenyl group optionally substituted by one or more substituents chosen, independently from one another, from halogen atoms, (C₁-C₆)alkyl or halo(C₁-C₆)alkyl groups;
Het represents an imidazole, isoxazole, indole, thiophene or pyridine group; and
R₂ represents one or more substituents chosen from a hydrogen atom, halogen atoms, halo(C₁-C₆)alkyl, (C₁-C₆)alkyl, phenyl, benzyl, (C₁-C₆)alkoxy or (C₁-C₆)alkylthio.

6. The compound of claim 1, wherein:
R₁ represents a phenyl group optionally substituted with one or more substituents chosen, independently from one another, from fluorine atoms, methyl or trifluoromethyl groups;
Het represents an imidazole, isoxazole, indole, thiophene or pyridine group; and
R₂ represents one or more substituents chosen from a hydrogen atom, chlorine atoms, methyl, methoxy, trifluoromethyl, methylthio, phenyl or benzyl groups.

7. The compound according to claim 1, wherein said compound is chosen from:
N-[(6-Azabicyclo[3.2.1]oct-5-yl)phenylmethyl](2,5-dichloro)thiophene-3-carboxamide;
N-[(6-Azabicyclo[3.2.1]oct-5-yl)phenylmethyl]-2-methylsulfanylnicotinamide;
N-[(6-Azabicyclo[3.2.1]oct-5-yl)phenylmethyl](3-chloro-4-trifluoromethyl)pyridine-2-carboxamide and its hydrochloride;
N-[(6-Azabicyclo[3.2.1]oct-5-yl)phenylmethyl](5-methyl-3-phenyl)isoxazole-4-carboxamide;
N-[(6-Azabicyclo[3.2.1]oct-5-yl)phenylmethyl](1-benzyl-2-ethyl-5-methoxy)-1H-indole-3-carboxamide;
[(6-Azabicyclo[3.2.1]oct-5-yl)phenylmethyl](1-benzyl)-1H-indole-4-carboxamide;
N-[(6-Azabicyclo[3.2.1]oct-5-yl)phenylmethyl](1-methyl)-1H-imidazole-4-carboxamide;
N-[(6-Azabicyclo[3.2.1]oct-5-yl)(4-fluorophenyl)methyl]-2-methylsulfanyl-nicotinamide and its hydrochloride;
N-[(6-Azabicyclo[3.2.1]oct-5-yl)(4-fluorophenyl)methyl](3-chloro-4-trifluoromethyl)-pyridine-2-carboxamide and its hydrochloride;
N-[(-6-Azabicyclo[3.2.1]oct-5-yl)-m-tolylmethyl]-2-methylsulfanylnicotinamide and its hydrochloride;
N-[(6-Azabicyclo[3.2.1]oct-5-yl)(3-trifluoromethylphenyl)methyl](3-chloro-4-trifluoromethyl)pyridine-2-carboxamide and its hydrochloride.

8. A process for preparing the compound of claim 1, comprising reacting a compound of general formula (II): 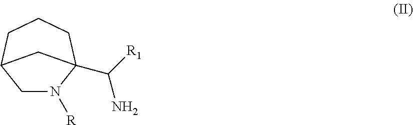
in which R and R₁ are as defined according to claim 1, with a compound of general formula (III): 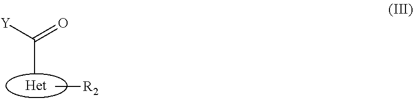
in which Y represents a leaving group or a chlorine atom and Het and R₂ are defined according to claim 1.

9. A compound of formula (II) 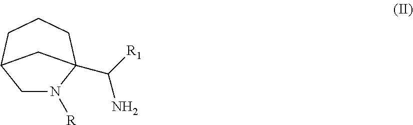
in which R and R₁ are defined according to claim 1.

10. A pharmaceutical composition comprising the compound of claim 1 or an addition salt of said compound with a pharmaceutically acceptable acid.

11. The pharmaceutical composition of claim 6 further comprising at least one pharmaceutically acceptable excipient.

12. A method of treating cognitive and/or behavioural disorders associated with neurodegenerative diseases or with dementia in a patient in need thereof comprising administering to said patient a therapeutically effective amount of the pharmaceutical composition of claim 10.

13. A method of treating psychoses, schizophrenia (deficit form and productive form) and acute or chronic neuroleptic-induced extrapyramidal symptoms in a patient in need thereof comprising administering to said patient a therapeutically effective amount of the pharmaceutical composition of claim 10.

14. A method of treating various forms of anxiety, panic attacks, phobias and obsessive-compulsive disorders in a patient in need thereof comprising administering to said patient a therapeutically effective amount of the pharmaceutical composition of claim 10.

15. A method of treating various forms of depression, including psychotic depression; for treating bipolar disorders, manic disorders and mood disorders; for treating disorders due to alcohol abuse or withdrawal, disorders of sexual behaviour, eating disorders and migraine disorders in a patient in need thereof comprising administering to said patient a therapeutically effective amount of the pharmaceutical composition of claim 10.

16. A method of treating pain in a patient in need thereof comprising administering to said patient a therapeutically effective amount of the pharmaceutical composition of claim 10.

17. A method of treating sleep disorders in a patient in need thereof comprising administering to said patient a therapeutically effective amount of the pharmaceutical composition of claim 10.

18. (canceled)

19. (canceled)

20. (canceled)

21. (canceled)

22. (canceled)

23. (canceled)
